(19) United States
(12) Patent Application Publication
(10) Pub. No.: US 2008/0139526 A1
(43) Pub. Date: Jun. 12, 2008

(54) Modified release dosage forms of amoxicillin

(76) Inventors: Donald Treacy, Woodbine, MD (US); Alan R. Potts, Damascus, MD (US); Henry H. Flanner, Montgomery Village, MD (US); Beth A. Burnside, Bethesda, MD (US)

Correspondence Address: Raymond E. Stauffer, Esq.;c/o Carella, Byrne, Bain, Gifillan, Cecchi,, Stewart & Olstein, 5 Becker Farm Road, Roseland, NJ, 07068, US

(21) Appl. No.: 11/634,633

(22) Filed: Dec. 6, 2006

(51) Int. Cl. A61K 31/43; A61P 31/04

(52) U.S. Cl. 514/197

(57) ABSTRACT

An amoxicillin product comprising: at least one modified release component(s), wherein the at least one modified release component(s) comprises at least amoxicillin and a pharmaceutically acceptable carrier; and wherein the product exhibits a mean in-vitro dissolution profile within a defined range characterized as follows: 1) the percent dissolved at 0.25 hours is between 25 and 55 percent; 2) the percent dissolved at 0.5 hours is between 30 and 60 percent; 3) the percent dissolved at 1 hour is between 50 and 85 percent; 4) the percent dissolved at 1.5 hours is between 70 and 95 percent; and 5) the percent dissolved at 2 hours is at least 85 percent.

This invention is directed to amoxicillin products, and to methods of use thereof.

In the treatment of bacterial infections amoxicillin is often dosed in immediate release (IR) formulations that may require multiple administrations over the course of any given 24-hour period. As is known in the art, such dosing regimens may be twice-a-day (b.i.d.), whereby the composition is administered every 12 hours; three times daily (t.i.d.), whereby the composition is administered every 8 hours; four times daily (q.i.d.), whereby the composition is administered every 6 hours; or such dosing regimens may even conceive of dosing the composition in excess of four administrations per day.

Amoxicillin is also available in a modified release formulation, e.g., as sold under the trademark AUGMENTIN XR.

The present invention is directed to an amoxicillin product that includes at least one modified release component which has a mean in-vitro dissolution profile when tested according to the method of Example 1, wherein at the following specified times, the percent dissolution of the total amoxicillin in the product is at least the specified minimum and does not exceed the specified maximum percent dissolution as follows:

| Time (hours) | Minimum (%) | Maximum (%) |
|---|---|---|
| 0.25 | 25 | 55 |
| 0.5 | 30 | 60 |
| 1 | 50 | 85 |
| 1.5 | 70 | 95 |
| 2 | 85 | — |

In one embodiment, the amoxicillin product has a mean in-vitro dissolution profile that falls within the following range of percent dissolution at the specified times:

| Time (hours) | Minimum (%) | Maximum (%) |
|---|---|---|
| 0.25 | 30 | 50 |
| 0.5 | 35 | 55 |
| 1 | 55 | 80 |
| 1.5 | 75 | 90 |
| 2 | 90 | — |

In another embodiment, the amoxicillin product has a mean in-vitro dissolution profile that falls within the following range of percent dissolution at the specified times:

| Time (hours) | Minimum (%) | Maximum (%) |
|---|---|---|
| 0.25 | 35 | 45 |
| 0.5 | 35 | 45 |
| 1 | 65 | 75 |
| 1.5 | 75 | 85 |
| 2 | 90 | — |

The mean in-vitro dissolution profile is determined in accordance with the dissolution method of Example 1. The mean results generated using the dissolution method of Example 1 would generally exhibit a % RSD (relative standard deviation) of less than 10%.

As a result, when the Specification and Claims, refer to a mean in-vitro dissolution profile of an amoxicillin product, the dissolution method of Example 1 is used to determine whether or not the amoxicillin product has the mean in-vitro dissolution profile that at the specified times falls within the specified minimum and maximum dissolution percentages.

It is to be understood that the in vivo dissolution profile of the amoxicillin product may or may not fall within the hereinabove described mean in-vitro dissolution profile ranges.

Although the Specification and Claims are not intended to be limited thereby, an amoxicillin product is formulated to have the herein described mean in-vitro dissolution profiles to extend the Tₘₐₓ of the amoxicillin product in-vivo and to thereby extend time over the minimum inhibitory concentration (MIC) in the plasma, while maintaining an acceptable area under the curve (AUC).

As used herein the term “amoxicillin” shall be broadly interpreted to include not only that active ingredient, but also all polymorphs, salts, and/or hydrates thereof.

An amoxicillin product that includes at least a modified release component that is formulated to have an in vitro dissolution profile as hereinabove described may be produced in a wide variety of forms and dosages of amoxicillin and may be administered in accordance with a variety of different protocols; for example, once-a-day, twice-a-day, three times a day.

In one embodiment, the product comprises a modified release component.

In one embodiment, the product includes an immediate release component and a delayed release component.

In another embodiment, the product includes an immediate release component and two or more delayed release components.

In another embodiment, the product includes one, two or three or more delayed release components and is free of an immediate release component.

In another embodiment, the product includes one, two or more extended (sustained) release components and is free of an immediate release component.

In a further embodiment, the product includes an immediate release component and one, two or more extended release components.

In yet another embodiment, the product includes an immediate release component and a combination of one or more delayed release components and one or more extended release components.

As used herein and as known in the art, an immediate release component is one in which the initiation of release, and/or the rate of release, of active ingredient is not substantially delayed, and/or slowed, and/or sustained, after administration of the product. As used herein and as known in the art, a modified release component is other than an immediate release component. Non-limiting examples of such modified release components include: delayed release component(s) which is one where after the delay the release is not sustained over a period of time, and a sustained (or extended) release component, which is one where release of active ingredient is sustained over a period of time and/or combinations of the foregoing. Immediate release, delayed released and sustained (extended) release components are components and terminology well known in the art and the formulation thereof is well within the skill of the art. The use of various combinations of the aforementioned components will be apparent to those of ordinary skill in the art in view of the disclosures herein, further guided by the disclosures of U.S. patent application Ser. Nos. 10/894,787; 10/894,786; 10/894,994; 10/917,059; 10/922,412; and 10/940,265; and by the disclosures of U.S. Pat. Nos. 6,544,555; 6,623,757; and 6,669,948; all of which are hereby incorporated by this reference in their entireties. In accordance with an embodiment of the invention, irrespective of the various components used in the amoxicillin product that includes at least one modified release, such components are formulated such that the amoxicillin product has a dissolution profile as hereinabove described.

In accordance with one embodiment of the invention, there are at least two components (at least one of which is a modified release component). One of the at least two components is an immediate release component, whereby initiation of release of the amoxicillin therefrom is not substantially delayed after administration of the amoxicillin composition, or is a delayed release component, whereby initiation of release of the amoxicillin therefrom is substantially delayed after administration of the amoxicillin composition. The second of the at least two components is a delayed release component (each delayed release component may be a pH sensitive or a non-pH sensitive delayed release component, depending on the type of amoxicillin composition), whereby the amoxicillin released therefrom is delayed until after initiation of release of the amoxicillin from the immediate release or first delayed release component. More particularly, the amoxicillin released from the second of the at least two components achieves a Cₘₐₓ (maximum concentration in the plasma) at a time after the amoxicillin released from the first of the at least two components achieves a Cₘₐₓ in the plasma.

In accordance with one embodiment of the invention, there are at least three components (at least one of which is a modified release component). One of the at least three components is an immediate release component whereby initiation of release of the amoxicillin therefrom is not substantially delayed after administration of the amoxicillin composition. The second and third of the at least three components are delayed release components (each of which may be a pH sensitive or a non-pH sensitive delayed release component, depending on the type of amoxicillin composition), whereby the amoxicillin released therefrom is delayed until after initiation of release of the amoxicillin from the immediate release component. More particularly, the amoxicillin released from the second of the at least three components achieves a Cₘₐₓ (maximum concentration in the plasma) at a time after the amoxicillin released from the first of the at least three components achieves a Cₘₐₓ in the plasma, and the amoxicillin released from the third component achieves a Cₘₐₓ in the plasma after the Cₘₐₓ of amoxicillin released from the second component.

In one embodiment, the second of the at least three components initiates release of the amoxicillin contained therein at least one hour after the first component, with the initiation of the release therefrom generally occurring no more than six hours after initiation of release of amoxicillin from the first component of the at least three components.

As hereinabove indicated, some embodiments of the amoxicillin composition may contain two, three, four, or more different components (provided that at least one is a modified release component).

In one three-component embodiment, the amoxicillin released from the third component reaches a Cₘₐₓ at a time later than the Cₘₐₓ is achieved for the amoxicillin released from each of the first and second components. In a preferred embodiment, release of amoxicillin from the third component is started after initiation of release of amoxicillin from both the first component and the second component. In one embodiment, Cₘₐₓ for amoxicillin released from the third component is achieved within eight hours.

In another three-component embodiment the release of amoxicillin from the second component may be contemporaneous with initiation of release of amoxicillin from the first component.

In another three-component embodiment the release of amoxicillin from the third component may be contemporaneous with initiation of release of amoxicillin from the second component.

In another embodiment, the amoxicillin composition may contain four components (at least one of which is a modified release component), with each of the four components having different release profiles, whereby the amoxicillin released from each of the four different components achieves a Cₘₐₓ at a different time.

In one preferred embodiment, the amoxicillin product contains at least two or at least three or at least four different components each with a different release profile, Cₘₐₓ for all the amoxicillin released from the amoxicillin product in those is achieved in less than twelve hours, and more generally is achieved in less than eleven hours.

In one embodiment, the amoxicillin product is a once-a-day composition, whereby after administration of the amoxicillin product, no further composition is administered during the day; i.e., the regimen is that the product is administered only once over a twenty-four hour period. Thus, in accordance with this embodiment, there is a single administration of an amoxicillin product with the amoxicillin being released in a manner such that overall amoxicillin release is effected with different release profiles in a manner such that the overall Cₘₐₓ for the amoxicillin composition is reached in less than twelve hours. The term single administration means that the total amoxicillin administered over a twenty-four hour period is administered at the same time, which can be a single dosage unit (tablet, capsule or sprinkle/sachet) or two or more thereof, provided that they are administered at essentially the same time.

In one embodiment, such once-a-day product is comprised of an immediate release component and two delayed release components wherein the first delayed release component initiates release of amoxicillin after release of amoxicillin from the immediate release component and the second delayed release component initiates release of amoxicillin after release of amoxicillin from the first delayed release component.

In a once-a-day amoxicillin product, such product has a dissolution profile as hereinabove described.

In one embodiment, the amoxicillin product is a twice-a-day product, whereby after an initial administration of the amoxicillin product, there is a further administration of the amoxicillin product at another time during the day; i.e., the regimen is that the composition is administered only twice over a twenty-four hour period.

In one embodiment, the twice-a-day amoxicillin product includes one component with the one component being a modified release component.

In one embodiment, the twice-a-day amoxicillin product includes two or more components with one of such two components being an immediate release component and the other of the two components being a modified release component.

In another embodiment the twice-a-day amoxicillin product contains one immediate release component and two or more modified release components, with a particular embodiment including two modified release components.

In a twice-a-day amoxicillin product, such product has a dissolution profile as hereinabove described.

Thus, in accordance with one embodiment, there is a b.i.d. administration of an amoxicillin product with the amoxicillin being released in a manner such that overall amoxicillin release is effected with different release profiles in a manner such that the overall Cₘₐₓ for each of the two administrations of the product is reached in less than twelve hours after each administration. The dose administered at each of the two administrations can be a single amoxicillin product or a plurality of amoxicillin products.

In one embodiment, the hereinabove described amoxicillin products having the hereinabove described in vitro dissolution profile have an extent of absorption (AUC_{o-inf}) that is at least 75% and in a preferred embodiment at least 80% of the extent of absorption (AUC_{o-inf}) of an amoxicillin product that provides for only immediate release of amoxicillin. In general, the extent of absorption (AUC_{o-inf}) of an amoxicillin product of the invention does not exceed the extent of absorption (AUC_{o-inf}) of an amoxicillin product that provides for only immediate release of amoxicillin. In comparing the products to determine extent of absorption, the AUC_{o-inf} is determined in either the fed state or the fasted state in accordance with FDA Guidance for Industry—Food-Effect Bioavailability and Fed Bioequivalence Studies, December 2002, and each of the products has the same amount of amoxicillin.

A “fasted state” for determining extent of absorption means that following an overnight fast of at least 10 hours, subjects should be administered the drug product with 240 mL (8 fluid ounces) of water. No food should be allowed for at least 4 hours post-dose. Water can be allowed as desired except for one hour before and after drug administration. Subjects should receive standardized meals scheduled at the same time in each period of the study.

Extent of absorption is generally determined by area under the curve (AUC). Two types of AUC are typically reported and are typically referred to as AUC₀₋ₜ, where the AUC is calculated over the range from time zero until the last plasma sample was taken, time t, and AUC_{0-inf} often denoted AUC_{inf} where the AUC_{t-inf} is calculated and added to AUC₀₋ₜ. AUC_{t-inf} is extrapolated from time t until the infinity point, which is the time point where the active ingredient concentration reaches 0 determined by extrapolation from the last measured concentration based on the elimination rate determined from the individual subject data.

When administering the amoxicillin product orally to a human, such product may be taken in the fed state or fasted state, preferably in the fed state.

As known in the art, oral administration of such a product to a human in a fed or fasted state has a meaning that is not limited by the FDA requirements for testing extent of absorption. In terms of administering a product to a human for use of a product, fed state means in conjunction with food (immediately prior to, with or immediately after intake of food). The fasted or non-fed state means other than in conjunction with the intake of food.

It is to be understood that when it is disclosed herein that a component initiates release after another component, such terminology means that the component is designed and is intended to produce such later initiated release. It is known in the art, however, notwithstanding such design and intent, that some “leakage” of antibiotic may occur. Such “leakage” is not “release” as used herein.

The amoxicillin product of the present invention, as hereinabove described, may be formulated for administration by a variety of routes of administration. For example, the amoxicillin composition may be formulated in a way that is suitable for topical administration; administration in the eye or the ear; rectal or vaginal administration; as a nasal preparation; by inhalation; as an injectable; or for oral administration. In a preferred embodiment, the amoxicillin composition is formulated in a manner such that it is suitable for oral administration.

For example, in formulating the amoxicillin product for topical administration, such as by application to the skin, the components, each of which contains amoxicillin, may be formulated for topical administration by including such components in an oil-in-water emulsion, or a water-in-oil emulsion. In such a formulation, an immediate release component may be in the continuous phase, and a delayed release component may be in a discontinuous phase. The formulation may also be produced in a manner for delivery of three components as hereinabove described. For example, there may be provided an oil-in-water-in-oil emulsion, with oil being a continuous phase that contains the immediate release component, water dispersed in the oil containing a first delayed release component, and oil dispersed in the water containing a third delayed release component.

It is also within the scope of the invention to provide an amoxicillin product in the form of a patch, which includes amoxicillin components having different release profiles, as hereinabove described.

In addition, the amoxicillin product may be formulated for use in the eye or ear or nose, for example, as a liquid emulsion. For example, the component may be coated with a hydrophobic polymer whereby a component is in the oil phase of the emulsion, and a component may be coated with hydrophilic polymer, whereby a component is in the water phase of the emulsion.

Furthermore, the amoxicillin product having at least one modified release component (whether or not combined with additional components to provide a plurality of different release profiles) may be formulated for rectal or vaginal administration, as known in the art. This may take the form of a cream, an emulsion, a suppository, or other dissolvable component similar to those used for topical administration.

The amoxicillin product may include an amount of amoxicillin from about 200 mg to about 2500 mg, depending on the form of the product. As non-limiting examples, the amoxicillin product may contain 475 mg or 775 mg or 1250 mg or 1550 mg or 2325 mg of amoxicillin.

In a preferred embodiment, the amoxicillin product is formulated in a manner suitable for oral administration. Thus, for example, for oral administration, each of the components may be used as a pellet or a particle, with a pellet or particle then being formed into a unitary pharmaceutical composition, for example, in a capsule, or embedded in a tablet, as a sprinkle, or suspended in a liquid for oral administration. In one non-limiting embodiment, the tablet may be a rapidly disintegrating tablet, whereby the various components of the product are released upon ingestion for further transport into the intestine in the form of pellets or granules.

Alternatively, in formulating an oral delivery system, each of the components of the composition may be formulated as a tablet, with each of the tablets being put into a capsule to produce a unitary amoxicillin product. Thus, as a non-limiting example, a three component amoxicillin product may include a first component in the form of a tablet that is an immediate release tablet, and may also include two or more additional tablets, each of which provides for a delayed release or a sustained release of the amoxicillin, as hereinabove described.

The amoxicillin product may be in the form of a sprinkle product; for example by placing the various components of the product in particulate form (for example as pellets) in a sachet, capsule or other form that can be used for administering the components in particulate form at the same time.

The formulation of an amoxicillin product including at least three components with different release profiles for different routes of administration is deemed to be within the skill of the art from the teachings herein. As known in the art, with respect to delayed release, the time of release can be controlled by a variety of mechanisms such as pH trigger point, coating thickness, choice of polymer, choice of plasticizer, osmotic pressure, physical swelling pressure and combinations of the foregoing.

In formulating an amoxicillin product in accordance with one embodiment of the invention, an immediate release component generally comprises about 45% of the total amoxicillin dose in the product, a first delayed release component generally comprises about 30% of the total amoxicillin dose in the product, and a second delayed release component generally comprises about 25% of the total amoxicillin dose in the product (all by weight). This embodiment is non-limiting, and when the disclosures herein are considered along with the entirety of the further knowledge that necessarily informs the level of ordinary skill in the art, the person of ordinary skill in the art will readily appreciate component percentages differing from those noted in the non-limiting embodiment, which percentages when combined to form an amoxicillin product has the hereinabove described mean in-vitro dissolution profile.

In accordance with an embodiment of the present invention, each of the components contains amoxicillin; however, each of the components may contain another antibiotic or other type of active ingredient.

In the embodiments hereinabove described, the amoxicillin product has an in vitro dissolution profile as hereinabove described.

### The Immediate Release Component

The immediate release portion of this system can be a mixture of ingredients that breaks down quickly after administration to release the amoxicillin. This can take the form of either a discrete tablet, pellet or granule that is mixed in with, or compressed with, the other components in the product.

In addition, it may be useful to have other ingredients in this system to aid in the dissolution of the drug, or the breakdown of the component after ingestion or administration. These ingredients can be surfactants, such as sodium lauryl sulfate, sodium monoglycerate, sorbitan monooleate, sorbitan monooleate, polyoxyethylene sorbitan monooleate, glyceryl monostearate, glyceryl monooleate, glyceryl monobutyrate, one of the non-ionic surfactants such as the Pluronic line of surfactants, or any other material with surface active properties, or any combination of the above.

### The Non-pH Sensitive Delayed Release Component

The components in this composition are the same as the immediate release unit, but with additional polymers integrated into the composition, or as coatings over the pellet or granule.

Several methods to affect a delayed release with non-pH dependent polymers are known to those skilled in the art. These include soluble or erodible barrier systems, enzymatically degraded barrier systems, rupturable coating systems, and plugged capsule systems among others. These systems have been thoroughly described in the literature (see “A Review of Pulsatile Drug Delivery” by Bussemer and Bodmeier in the Winter 2001 issue of American Pharmaceutical Review) and formulations and methods for their manufacture are hereby incorporated by reference.

Materials that can be used to obtain a delay in release suitable for this component of the invention can be, but are not limited to, polyethylene glycol (PEG) with molecular weight above 4,000 daltons (Carbowax, Polyox), waxes such as white wax or bees wax, paraffin, acrylic acid derivatives (Eudragit), propylene glycol, and ethylcellulose.

Typically these materials can be present in the range of 0.5-40% (W/W) of this component.

### The pH Sensitive (Enteric) Release Component

The components in this composition are the same as the immediate release component, but with additional polymers integrated into the composition, or as coatings over the pellet or granule to delay release.

The kind of materials useful for this purpose can be, but are not limited to, cellulose acetate pthalate, Eudragit L, Eudragit S, Eudragit FS, and other pthalate salts of cellulose derivatives.

These materials can be present in concentrations from 4-30% (W/W).

### Sustained Release Component

The components in this composition are the same as the immediate release component, but with additional polymers integrated into the composition, or as coatings over a tablet, pellet or granule to provide a sustained release of the pharmaceutical.

The kind of materials useful for this purpose can be, but are not limited to, ethylcellulose; hydroxypropylmethylcellulose; hydroxypropylcellulose; hydroxyethylcellulose; carboxymethylcellulose; methylcellulose; nitrocellulose; Eudragit R; Eudragit RS; and Eudragit RL; Carbopol; or polyethylene glycols with molecular weights in excess of 8,000 daltons.

These materials can be present in concentrations from 4-40% (W/W).

When it is desired to delay initiation of release of the sustained release component, an appropriate coating may be used to delay initiation of the sustained release, such as a pH sensitive or a non-pH sensitive coating.

### The Non-pH Sensitive Coating for Sustained Release Component

Materials that can be used to obtain a delay in release suitable for this component of the invention can be, but are not limited to, polyethylene glycol (PEG) with molecular weight above 4,000 daltons (Carbowax, Polyox), waxes such as white wax or bees wax, paraffin, acrylic acid derivatives (Eudragit RS), cellulose acetate, and ethylcellulose.

Typically these materials can be present in the range of 0.5-25% (W/W) of this component. Preferably the materials are present in an amount just enough to provide the desired in vivo lag time and Tₘₐₓ.

### The pH Sensitive Coating for Sustained Release Component

The kind of materials useful for this purpose can be, but are not limited to, cellulose acetate pthalate, Eudragit L, Eudragit S, Eudragit FS, and other pthalate salts of cellulose derivatives.

These materials can be present in concentrations from 4-30% (W/W) or more. Preferably the materials are present in an amount just enough to provide the desired in vivo lag time and Tₘₐₓ.

As hereinabove indicated, the units comprising the amoxicillin composition of the present invention can be in the form of discrete pellets or particles contained in a capsule, particles embedded in a tablet, as sprinkles or suspended in a liquid suspension.

The amoxicillin products of the present invention may be administered, for example, by any of the following routes of administration: sublingual, transmucosal, transdermal, parenteral, etc., and preferably are administered orally. The product includes a therapeutically effective amount of the amoxicillin, which amount will vary with the disease or infection to be treated, and the number of times that the product is to be delivered in a day. The product is administered to a patient or subject (i.e., a human or an animal) in an amount effective for treating a bacterial infection.

In accordance with one embodiment, the amoxicillin product has an overall release profile such that when administered to a human the maximum plasma concentration of the total amoxicillin released from the product is reached in less than twelve hours, preferably in less than eleven hours.

In a further aspect, the present invention provides a method of treating various infections in a human, caused by bacterial pathogens, which treating comprises administering to the patient, or to the subject, the herein described amoxicillin product. As non-limiting examples of the indications for which the amoxicillin product may be used to treat a patient there may be mentioned: pharyngitis, tonsillitis, sinusitis, bronchitis, pneumoniae, ear infection (otitis media), uncomplicated skin and skin structure infections, and uncomplicated urinary infections.

As non-limiting examples of the infectious bacterial pathogens against which the amoxicillin products may be used, there may be mentioned Gram-Positive Aerobes such as Staphylococcus aureus, Enterococcus faecalis, Staphylococcus epidermidis, Staphylococcus saprophyticus, Streptococcus pneumoniae, Streptococcus pyogenes, and viridans group Streptococcus; Gram-Negative Aerobes such as Enterobacter species, Escherichia coli, Haemophilus influenzae, Klebsiella species, Moraxella catarrhalis, Eikenella corrodens, Neisseria gonorrhoeae, and Proteus mirabilis; Anaerobic Bacteria such as Bacteroides species, including Bacteroides fragilis, Fusobacterium species, and Peptostreptococcus species.

In one embodiment the amoxicillin product is formulated to specifically target tonsillopharyngitis secondary to Streptococcus pyogenes.

It will be appreciated by those of ordinary skill in the art that the methods and formulations described for the amoxicillin products are also applicable to amoxicillin in combination with clavulanate, or in combination with other beta-lactamase inhibitors, particularly for treating infections where beta-lactamase producing pathogens are implicated as the primary infection or as a co-infection.

In treating a bacterial infection, the amoxicillin product is formulated to provide a concentration of amoxicillin in the plasma that is above the MIC of the bacterial pathogen for a period of time each day that is effective for treating the bacterial infection. The amoxicillin product is administered for a number of days that provides a concentration of MIC in the plasma for a total time over MIC (daily time over MIC multiplied by the number of days of treatment) that is effective for treating the bacterial infection.

The invention will be further described with respect to the following Examples; however, the scope of the invention is not limited by such Examples. Unless otherwise specified, parts and percentages are by weight.

### EXAMPLE 1

Dissolution Method

The drug product dissolution rate is measured using USP Apparatus II (paddles) with a paddle speed of 75 rpm at 37° C. This procedure generally follows the procedure in USP General Chapter <711>—Dissolution. Additional parameters for execution of this test method are described below. The dissolution media used initially is 0.05 M phosphate buffer at a pH of approximately 2.0. After the dissolution has been run for 30 minutes at a pH of approximately 2.0, the pH of the media is adjusted to approximately 6.0 over approximately 5 minutes with a 5 M KOH solution. After the pH is stabilized at approximately 6.0, the pH of the media is linearly increased over a period of 2.5 hours to a pH endpoint of approximately 7.8 using a 0.5 M KOH solution. After reaching the pH endpoint for the dissolution, the paddles are allowed to run for an additional 30 minutes. Samples are pulled at 15 minutes, 30 minutes, 60 minutes, 90 minutes, and 210 minutes. Samples are analyzed using a UV/VIS spectrophotometer using an external standard at a wavelength of 230 nm.

### EXAMPLE 2

### Formulation

An amoxicillin tablet (Applicants' MP) is made that incorporates an immediate release component (Pulse 1); a first delayed release component (Pulse 2) and a second delayed release component (Pulse 3). The tablet rapidly disintegrates upon ingestion.

1. Product Description

The Applicants' MP Tablet, 775 mg, is a three-pulse dosage form. The tablet is manufactured by combining the immediate-release granulation (Pulse 1, 45%) with two functionally coated delayed-release pellets (Pulse 2, 30% and Pulse 3, 25%). A non-functional, protective film is then applied to the tablet.

The qualitative composition, the pharmaceutical grade and the function of the individual components comprising each dosage form are listed in Table 1-1.

TABLE 1-1
Qualitative Composition of Applicants' MP Tablet, 775 mg

| Component & Grade | Manufacturer | Function |
|---|---|---|
| Amoxicillin, USP | Fersinsa | API |
| Microcrystalline Cellulose, NF (Avicel PH101) | FMC | Diluent |
| Silicified Microcrystalline Cellulose (Prosolv SMCC 90) | JRS Pharma | Diluent |
| Polyoxyl 35 Castor Oil, NF (Cremophor EL) | BASF | Wetting agent |
| Povidone, USP (Kollidon 30) | BASF | Binder |
| Crospovidone, NF (Polyplasdone XL) | ISP | Disintegrant |
| Magnesium Stearate, NF | Mallinckrodt | Lubricant |
| Hypromellose Acetate Succinate, NF (AQOAT AS-HF) | Shin-Etsu | Functional film coat |
| Methacrylic Acid Copolymer Dispersion, NF (Eudragit | Rohm | Functional film coat |
| L30D-55) | | |
| Triethyl Citrate, NF | Morflex | Plasticizer |
| Talc, USP (Imperial 1885L) | Luzenac America | Antitacking agent |
| Sodium Lauryl Sulfate, NF | Spectrum | Surfactant |
| Opadry ® Clear YS-1-19025-A | Colorcon | Non-functional film |
| | | coat |
| Opadry ® Blue 03B10826 | Colorcon | Non-functional film |
| | | coat |

2. Quantitative Composition of Applicants' MP Tablet, 775 mg

The quantitative composition for Applicants' MP Tablet, 775 mg, amoxicillin granules, amoxicillin core pellets, and pulsatile pellets are located in Tables 2-1 through 2-5.

2.1 Applicants' MP Tablet, 775 mg

A batch of 144.9 kg of Applicants' MP Tablet, 775 mg, containing 891.2 mg of amoxicillin trihydrate per tablet, equivalent to 775 mg amoxicillin is manufactured. The total tablet weight is approximately 1.5 grams. The quantitative composition for Applicants' MP Tablet, 775 mg is listed below in Table 2-1.

TABLE 2-1
Quantitative Composition of Applicants' MP Tablet, 775 mg

| Component | w/w % |
|---|---|
| Amoxicillin, USP | 59.5 |
| Silicified Microcrystalline Cellulose (Prosolv | 20.8 |
| SMCC 90) | |
| Crospovidone, NF (Polyplasdone XL) | 3.9 |
| Methacrylic Acid Copolymer Dispersion, NF | 2.9* |
| (Eudragit L30D-55) | |
| Opadry ® Blue 03B10826 | 2.4 |
| Talc, USP (Imperial 1885L) | 2.0 |
| HypromelloseAcetate Succinate, NF (AQOAT | 1.9 |
| AS-HF) | |
| Microcrystalline Cellulose, NF (Avicel PH101) | 1.8 |
| Povidone, USP (PVP K30) | 1.5 |
| Opadry ® Clear YS-1-19025-A | 1.0 |
| Magnesium Stearate, NF | 1.0 |
| Triethyl Citrate, NF | 0.9 |
| Polyoxyl 35 Castor Oil, NF (Cremophor EL) | 0.3 |
| Sodium Lauryl Sulfate, NF | 0.1 |
| Opacode ® Black S-1-17734 | 0.0 |
| Total | 100.0 |

*Weight percent of solids content

2.2 Amoxicillin Trihydrate (97%) Granules

A batch of 20 kg of Amoxicillin Trihydrate (97%) Granules is manufactured. The Amoxicillin granules serve as Pulse 1 of the final formulation. The granules are compressed with Pulse 2 and 3 pellets and other inactive components to form the tablet core.

A standard wet granulation process known to one skilled in the art is used for preparation of the Amoxicillin Granules. The wet granules are discharged and fed into a Dome Extrusion Granulator. The wet extruded granules are then dried for a fixed period of time or until the LOD (loss on drying) of the granules is suitable for the formulation, typically less than 15%. The dried granules are then sized in a Rotating Impeller Screening Mill. The milled material is collected into drums. The quantitative composition for Amoxicillin Trihydrate (97%) Granules is listed below in Table 2-2.

TABLE 2-2
Quantitative Composition of Amoxicillin Trihydrate (97%) Granules
(Used for Pulse 1 in compression blend)

| Component | w/w % |
|---|---|
| Amoxicillin, USP | 97.0 |
| Povidone, USP | 3.0 |
| (Kollidon 30) | |
| Total | 100.0 |

2.3 Amoxicillin Trihydrate (92%) Core Pellets

A batch of 20 kg Amoxicillin Trihydrate (92%) Core Pellets is manufactured. The Amoxicillin core pellets are coated with functional film coating to produce Pulse 2 and 3 pellets.

The core pellets are prepared using the unit operations of wet granulating, extruding, spheronizing, fluid bed drying and sizing. The quantitative composition for Amoxicillin Trihydrate (92%) Core Pellets is listed below in Table 2-3.

TABLE 2-3
Quantitative Composition of Amoxicillin Trihydrate (92%) Core Pellets
(Used for Amoxicillin Pulse 2 and 3 Pellets)

| Component | w/w % |
|---|---|
| Amoxicillin, USP | 92.0 |
| Microcrystalline Cellulose, NF | 5.0 |
| (Avicel PH 101) | |
| Povidone, USP | 2.0 |
| (Kollidon 30) | |
| Polyoxyl 35 Castor Oil, NF (Cremophor EL) | 1.0 |
| Total | 100.0 |

2.4 Amoxicillin Trihydrate (76.7%) Pulse 2 Pellets

A 16.8 kg batch of Amoxicillin Trihydrate (76.7%) Pulse 2 Pellets is manufactured by applying a 20% total solids weight gain of Eudragit L30D-55 to 14.0 kg of the Amoxicillin Trihydrate (92%) Core Pellets.

The Pulse 2 Pellets are prepared by coating the previously prepared Core Pellets with a functional film coat of methacrylic acid copolymer dispersion, 20% w/w. Prior to the coating process, a dispersion of the methacrylic acid copolymer is made according to the manufacturer's instructions. The dispersion is applied to the Amoxicillin Core pellets using a Fluid Bed Bottom Spray Coater, equipped with appropriate spray nozzles and a fixed column gap distance.

The pellets are then appropriately sized. The Amoxicillin Pulse 2 Pellets may be held in ambient warehouse conditions until further processing. The quantitative composition for Amoxicillin Trihydrate (76.7%) Pulse 2 Pellets is listed below in Table 2-4.

TABLE 2-4
Quantitative Composition of Amoxicillin Trihydrate
(76.7%) Pulse 2 Pellets

| Component | w/w % |
|---|---|
| Amoxicillin, USP | 76.7 |
| Microcrystalline Cellulose, NF | 4.2 |
| (Avicel PH 101) | |
| Polyoxyl 35 Castor Oil, NF (Cremophor EL) | 0.8 |
| Povidone, USP (Kollidon 30) | 1.7 |
| Methacrylic Acid Copolymer Dispersion, NF | 10.4* |
| (Eudragit L30D-55) | |
| Talc, USP | 5.2 |
| Triethyl Citrate, NF | 1.0 |
| Total | 100 |

*Weight percent of solids content

2.5 Amoxicillin Trihydrate (76.0%) Pulse 3 Pellets

A 12.5 kg batch of Amoxicillin Trihydrate (73.6%) Pulse 3 Pellets is manufactured by applying a 5% total solids wt gain sub-coat of Eudragit L30D-55 and an over-coat of 20% total solids weight gain of AQOAT AS-HF to 10.0 kg of the Amoxicillin Trihydrate (92%) Core Pellet.

Prior to the subcoating process, a dispersion of the methacrylic acid copolymer is made according to the manufacturer's instructions. The second coating material is prepared according to the manufacturer's instructions. The subcoat layer is then applied to the Amoxicillin Core Pellets using the same Fluid Bed Bottom Spray Coater as used for preparation of the Pulse 2 Pellets.

The second coating dispersion is then immediately applied to the sub-coated pellets still in the Fluid Bed Bottom Spray Coater. The atomization air used for the second coating process is set at the same pressure as used for the sub coating process. The coating process is complete when all of the dispersion has been applied. Following a drying period the final coated pellets are cooled. The quantitative composition for Amoxicillin Trihydrate (73.6%) Pulse 3 Pellets is listed below in Table 2-5.

TABLE 2-5
Quantitative Composition of Amoxicillin Trihydrate
(73.6%) Pulse 3 Pellets

| Component | w/w % |
|---|---|
| Amoxicillin, USP | 73.6 |
| Microcrystalline Cellulose, NF | 4.0 |
| (Avicel PH 101) | |
| Polyoxyl 35 Castor Oil, NF (Cremophor EL) | 0.8 |
| Povidone, USP | 1.6 |
| (Kollidon 30) | |
| Methacrylic Acid Copolymer Dispersion, NF (Eudragit | 2.5* |
| L30D-55) | |
| Hypromellose Acetate Succinate, NF | 9.6 |
| (AQOAT AS-HF) | |
| Talc, USP | 4.1 |
| Triethyl Citrate, NF | 3.5 |
| Sodium Lauryl Sulfate, NF | 0.3 |
| Total | 100 |

*Weight percent of solids content

For example, the above product may be used to treat tonsillopharyngitis secondary to Streptococcus pyogenes by administering such product to a human once-a-day for 10 days.

### EXAMPLE 3

The core pellets of Part 2.3 of Example 2 are coated with a non-functional immediate release film coating to produce Pulse 1 pellets. The Pulse 1 pellets as well as Pulse 2 and Pulse 3 pellets of Example 2 are used as a sprinkle product by placing the Pulse 1, Pulse 2 and Pulse 3 pellets in a sachet, capsule or other form that can be used for simultaneous delivery of the three pulses in a particulate form. In one embodiment, Pulse 1, Pulse 2 and Pulse 3 are combined to provide 45%, 30% and 25% of Pulse 1, Pulse 2, and Pulse 3, respectively.

Such combination of Pulses 1, 2 and 3 may be formulated into a sprinkle product; e.g., a twice-a-day product that contains 475 mg or 775 mg of amoxicillin. In another embodiment, Pulse 1, 2 and 3 may be combined into a once-a-day sprinkle product that contains 775 mg or 1250 mg, or 1550 mg, or 2325 mg of amoxicillin. The sprinkle product may be sprinkled over applesauce, yogurt, or other soft food for administration. The product should not be chewed or crushed.

### EXAMPLE 4

The amoxicillin product of Example 2 was tested using the procedure of Example 1.

The dissolution profile was as follows:

| Time (hours) | % Dissolution |
|---|---|
| 0.25 | 37 |
| 0.5 | 39 |
| 1 | 68 |
| 1.5 | 82 |
| 2 | 92 |

Numerous modifications and variations of the present invention are possible in light of the above teachings; therefore, except as set forth in the claims the invention is not limited to described embodiments.

1. An amoxicillin product comprising: at least one modified release component(s) that includes amoxicillin, said product having a mean in-vitro dissolution profile as determined by the method of Example 1 herein wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 25 | 55 |
| 0.5 | 30 | 60 |
| 1 | 50 | 85 |
| 1.5 | 70 | 95 |
| 2 | 85 | — |

2. The product of claim 1, wherein said product further comprises an immediate release component.

3. The product of claim 1, wherein said modified release component is selected from the group consisting of: a delayed release component, sustained release component, and combinations of the foregoing.

4. The product of claim 3, wherein said modified release component is a delayed release component.

5. The product of claim 3, wherein said modified release component is a sustained release component.

6. The product of claim 1, wherein the product is a twice-a-day product.

7. The product of claim 1, wherein the product is a once-a-day product that includes the daily dosage of amoxicillin.

8. The product of claim 1, wherein the product contains about from about 200 mg to about 2500 mg of amoxicillin.

9. The product of claim 1, wherein the product contains about 775 mg of amoxicillin.

10. An amoxicillin product comprising an immediate release component that includes amoxicillin, a first delayed release component that includes amoxicillin and initiates release of amoxicillin after the immediate release component, a second delayed release component that includes amoxicillin and initiates release of amoxicillin after the first delayed release component, said amoxicillin product having a mean in-vitro dissolution profile as determined by the method of Example 1 wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 25 | 55 |
| 0.5 | 30 | 60 |
| 1 | 50 | 85 |
| 1.5 | 70 | 95 |
| 2 | 85 | — |

11. The product of claim 10, wherein the product is a twice-a-day product.

12. The product of claim 10, wherein the product is a once-a-day product that includes the daily dosage of amoxicillin.

13. The product of claim 10, wherein the product contains about from about 200 mg to about 2500 mg of amoxicillin.

14. The product of claim 10, wherein the product contains about 775 mg of amoxicillin.

15. An amoxicillin product comprising: at least one modified release component(s) that includes amoxicillin, said product having a mean in-vitro dissolution profile as determined by the method of Example 1 herein wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 25 | 55 |
| 0.5 | 30 | 60 |
| 1 | 50 | 85 |
| 1.5 | 70 | 95 |
| 2 | 85 | — |

and wherein said product when administered as a single dose in either the fed state or the fasted state has an AUC_{0-inf} of at least 75% of the AUC_{0-inf} of an immediate release amoxicillin product, wherein the AUC_{0-inf} of said immediate release amoxicillin product is determined in either the fed state or the fasted state.

16. An amoxicillin product comprising: at least one modified release component(s) that includes amoxicillin, said product having a mean in-vitro dissolution profile as determined by the method of Example 1 herein wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 30 | 50 |
| 0.5 | 35 | 55 |
| 1 | 55 | 80 |
| 1.5 | 75 | 90 |
| 2 | 90 | — |

17. The product of claim 16, wherein said product further comprises an immediate release component.

18. The product of claim 16, wherein said modified release component is selected from the group consisting of: a delayed release component, sustained release component, and combinations of the foregoing.

19. The product of claim 18, wherein said modified release component is a delayed release component.

20. The product of claim 18, wherein said modified release component is a sustained release component.

21. The product of claim 16, wherein the product is a twice-a-day product.

22. The product of claim 16, wherein the product is a once-a-day product that includes the daily dosage of amoxicillin.

23. The product of claim 16, wherein the product contains about from about 200 mg to about 2500 mg of amoxicillin.

24. The product of claim 16, wherein the product contains about 775 mg of amoxicillin.

25. An amoxicillin product comprising an immediate release component that includes amoxicillin, a first delayed release component that includes amoxicillin and initiates release of amoxicillin after the immediate release component, a second delayed release component that includes amoxicillin and initiates release of amoxicillin after the first delayed release component, said amoxicillin product having a mean in-vitro dissolution profile as determined by the method of Example 1 wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 30 | 50 |
| 0.5 | 35 | 55 |
| 1 | 55 | 80 |
| 1.5 | 75 | 90 |
| 2 | 90 | — |

26. The product of claim 25, wherein the product is a twice-a-day product.

27. The product of claim 25, wherein the product is a once-a-day product that includes the daily dosage of amoxicillin.

28. The product of claim 25, wherein the product contains about from about 200 mg to about 2500 mg of amoxicillin.

29. The product of claim 25, wherein the product contains about 775 mg of amoxicillin.

30. An amoxicillin product comprising: at least one modified release component(s) that includes amoxicillin, said product having a mean in-vitro dissolution profile as determined by the method of Example 1 herein wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 30 | 50 |
| 0.5 | 35 | 55 |
| 1 | 55 | 80 |
| 1.5 | 75 | 90 |
| 2 | 90 | — |

and wherein said product when administered as a single dose in either the fed state or the fasted state has an AUC_{0-inf} of at least 75% of the AUC_{0-inf} of an immediate release amoxicillin product, wherein the AUC_{0-inf} of said immediate release amoxicillin product is determined in either the fed state or the fasted state.

31. An amoxicillin product comprising: at least one modified release component(s) that includes amoxicillin, said product having a mean in-vitro dissolution profile as determined by the method of Example 1 herein wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 35 | 45 |
| 0.5 | 35 | 45 |
| 1 | 65 | 75 |
| 1.5 | 75 | 85 |
| 2 | 90 | — |

32. The product of claim 31, wherein said product further comprises an immediate release component.

33. The product of claim 31, wherein said modified release component is selected from the group consisting of: a delayed release component, sustained release component, and combinations of the foregoing.

34. The product of claim 33, wherein said modified release component is a delayed release component.

35. The product of claim 33, wherein said modified release component is a sustained release component.

36. The product of claim 31, wherein the product is a twice-a-day product.

37. The product of claim 31, wherein the product is a once-a-day product that includes the daily dosage of amoxicillin.

38. The product of claim 31, wherein the product contains about from about 200 mg to about 2500 mg of amoxicillin.

39. The product of claim 31, wherein the product contains about 775 mg of amoxicillin.

40. An amoxicillin product comprising an immediate release component that includes amoxicillin, a first delayed release component that includes amoxicillin and initiates release of amoxicillin after the immediate release component, a second delayed release component that includes amoxicillin and initiates release of amoxicillin after the first delayed release component, said amoxicillin product having a mean in-vitro dissolution profile as determined by the method of Example 1 wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 35 | 45 |
| 0.5 | 35 | 45 |
| 1 | 65 | 75 |
| 1.5 | 75 | 85 |
| 2 | 90 | — |

41. The product of claim 40, wherein the product is a twice-a-day product.

42. The product of claim 40, wherein the product is a once-a-day product that includes the daily dosage of amoxicillin.

43. The product of claim 40, wherein the product contains about from about 200 mg to about 2500 mg of amoxicillin.

44. The product of claim 40, wherein the product contains about 775 mg of amoxicillin.

45. An amoxicillin product comprising: at least one modified release component(s) that includes amoxicillin, said product having a mean in-vitro dissolution profile as determined by the method of Example 1 herein wherein at the following times the percent dissolution of the total amoxicillin of the product is no less than the following minimum percent dissolution and no greater than the following maximum percent specified for such times:

| Time (hours) | Minimum (%) | Maximum (%) |
|---|---|---|
| 0.25 | 35 | 45 |
| 0.5 | 35 | 45 |
| 1 | 65 | 75 |
| 1.5 | 75 | 85 |
| 2 | 90 | — |

and wherein said product when administered as a single dose in either the fed state or the fasted state has an AUC_{0-inf} of at least 75% of the AUC_{0-inf} of an immediate release amoxicillin product, wherein the AUC_{0-inf} of said immediate release amoxicillin product is determined in either the fed state or the fasted state.

46. A process for treating a bacterial infection in a patient or a subject comprising: administering to a patient or a subject the product of claim 1.

47. A process for treating a bacterial infection in a patient or a subject comprising: administering to a patient or a subject the product of claim 2.

48. A process for treating a bacterial infection in a patient or a subject comprising: administering to a patient or a subject the product of claim 3.

49. A process for treating a bacterial infection in a patient or a subject comprising: administering to a patient or a subject the product of claim 4.

50. A process for treating a bacterial infection in a patient or a subject comprising: administering to a patient or a subject the product of claim 5.

51. A process for treating a bacterial infection in a patient or a subject comprising: administering to a patient or a subject the product of claim 6.

52. A process for treating a bacterial infection in a patient or a subject comprising: administering to a patient or a subject the product of claim 7.

53. A process for treating a bacterial infection in a patient or a subject comprising: administering to a patient or a subject the product of claim 8.

54. A process for treating a bacterial infection in a patient or a subject comprising: administering to a patient or a subject the product of claim 9.

55. A process for treating a bacterial infection in a patient or a subject comprising: administering to a patient or a subject the product of claim 10.

56. A process for treating a bacterial infection in a patient or a subject comprising: administering to a patient or a subject the product of claim 11.

57. A process for treating a bacterial infection in a patient or a subject comprising: administering to a patient or a subject the product of claim 12.

58. A process for treating a bacterial infection in a patient or a subject comprising: administering to a patient or a subject the product of claim 13.

59. A process for treating a bacterial infection in a patient or a subject comprising: administering to a patient or a subject the product of claim 14.

60. A process for treating a bacterial infection in a patient or a subject comprising: administering to a patient or a subject the product of claim 15.

61. A process for treating a bacterial infection in a patient or a subject comprising: administering to a patient or a subject the product of claim 16.

62. A process for treating a bacterial infection in a patient or a subject comprising: administering to a patient or a subject the product of claim 17.

63. A process for treating a bacterial infection in a patient or a subject comprising: administering to a patient or a subject the product of claim 18.

64. A process for treating a bacterial infection in a patient or a subject comprising: administering to a patient or a subject the product of claim 19.

65. A process for treating a bacterial infection in a patient or a subject comprising: administering to a patient or a subject the product of claim 20.

66. A process for treating a bacterial infection in a patient or a subject comprising: administering to a patient or a subject the product of claim 21.

67. A process for treating a bacterial infection in a patient or a subject comprising: administering to a patient or a subject the product of claim 22.

68. A process for treating a bacterial infection in a patient or a subject comprising: administering to a patient or a subject the product of claim 23.

69. A process for treating a bacterial infection in a patient or a subject comprising: administering to a patient or a subject the product of claim 24.

70. A process for treating a bacterial infection in a patient or a subject comprising: administering to a patient or a subject the product of claim 25.

71. A process for treating a bacterial infection in a patient or a subject comprising: administering to a patient or a subject the product of claim 26.

72. A process for treating a bacterial infection in a patient or a subject comprising: administering to a patient or a subject the product of claim 27.

73. A process for treating a bacterial infection in a patient or a subject comprising: administering to a patient or a subject the product of claim 28.

74. A process for treating a bacterial infection in a patient or a subject comprising: administering to a patient or a subject the product of claim 29.

75. A process for treating a bacterial infection in a patient or a subject comprising: administering to a patient or a subject the product of claim 30.

76. A process for treating a bacterial infection in a patient or a subject comprising: administering to a patient or a subject the product of claim 31.

77. A process for treating a bacterial infection in a patient or a subject comprising: administering to a patient or a subject the product of claim 32.

78. A process for treating a bacterial infection in a patient or a subject comprising: administering to a patient or a subject the product of claim 33.

79. A process for treating a bacterial infection in a patient or a subject comprising: administering to a patient or a subject the product of claim 34.

80. A process for treating a bacterial infection in a patient or a subject comprising: administering to a patient or a subject the product of claim 35.

81. A process for treating a bacterial infection in a patient or a subject comprising: administering to a patient or a subject the product of claim 36.

82. A process for treating a bacterial infection in a patient or a subject comprising: administering to a patient or a subject the product of claim 37.

83. A process for treating a bacterial infection in a patient or a subject comprising: administering to a patient or a subject the product of claim 38.

84. A process for treating a bacterial infection in a patient or a subject comprising: administering to a patient or a subject the product of claim 39.

85. A process for treating a bacterial infection in a patient or a subject comprising: administering to a patient or a subject the product of claim 40.

86. A process for treating a bacterial infection in a patient or a subject comprising: administering to a patient or a subject the product of claim 41.

87. A process for treating a bacterial infection in a patient or a subject comprising: administering to a patient or a subject the product of claim 42.

88. A process for treating a bacterial infection in a patient or a subject comprising: administering to a patient or a subject the product of claim 43.

89. A process for treating a bacterial infection in a patient or a subject comprising: administering to a patient or a subject the product of claim 44.

90. A process for treating a bacterial infection in a patient or a subject comprising: administering to a patient or a subject the product of claim 45.

91. An amoxicillin product comprising at least one modified release dosage component, said product having an equivalent extent of absorption when administered in both the fasted and fed states.

92. A process for treating a bacterial infection in a patient or a subject comprising: administering to a patient or a subject the product of claim 91.

93. The product of claim 1, further comprising clavulanate or other beta-lactamase inhibitor.

94. The product of claim 10, further comprising clavulanate or other beta-lactamase inhibitor.

95. The product of claim 15, further comprising clavulanate or other beta-lactamase inhibitor.

96. The product of claim 16, further comprising clavulanate or other beta-lactamase inhibitor.

97. The product of claim 25, further comprising clavulanate or other beta-lactamase inhibitor.

98. The product of claim 30, further comprising clavulanate or other beta-lactamase inhibitor.

99. The product of claim 31, further comprising clavulanate or other beta-lactamase inhibitor.

100. The product of claim 40, further comprising clavulanate or other beta-lactamase inhibitor.

101. The product of claim 45, further comprising clavulanate or other beta-lactamase inhibitor.

102. The product of claim 91, further comprising clavulanate or other beta-lactamase inhibitor.

103. A process for treating a bacterial infection in a patient or a subject comprising: administering to a patient or a subject the product of claim 93.

104. A process for treating a bacterial infection in a patient or a subject comprising: administering to a patient or a subject the product of claim 94.

105. A process for treating a bacterial infection in a patient or a subject comprising: administering to a patient or a subject the product of claim 95.

106. A process for treating a bacterial infection in a patient or a subject comprising: administering to a patient or a subject the product of claim 96.

107. A process for treating a bacterial infection in a patient or a subject comprising: administering to a patient or a subject the product of claim 97.

108. A process for treating a bacterial infection in a patient or a subject comprising: administering to a patient or a subject the product of claim 98.

109. A process for treating a bacterial infection in a patient or a subject comprising: administering to a patient or a subject the product of claim 99.

110. A process for treating a bacterial infection in a patient or a subject comprising: administering to a patient or a subject the product of claim 100.

111. A process for treating a bacterial infection in a patient or a subject comprising: administering to a patient or a subject the product of claim 101.

112. A process for treating a bacterial infection in a patient or a subject comprising: administering to a patient or a subject the product of claim 102.
